(19) United States
(12) Patent Application Publication
(10) Pub. No.: US 2018/0117042 A1
(43) Pub. Date: May 3, 2018

(54) METHODS FOR TREATING RESPIRATORY SYNCYTIAL VIRUS INFECTION

(71) Applicant: ALIOS BIOPHARMA, INC., South San Francisco, CA (US)

(72) Inventors: Sushmita Mukherjee CHANDA, Redwood City, CA (US); John Fry, San Francisco, CA (US); Lawrence M. BLATT, Healdsburg, CA (US)

(21) Appl. No.: 15/795,079

(22) Filed: Oct. 26, 2017

Related U.S. Application Data
(60) Provisional application No. 62/413,801, filed on Oct. 27, 2016.

(51) Int. Cl. A61K 31/506; A61P 31/14

(52) U.S. Cl. CPC A61K 31/506; A61P 31/14

(57) ABSTRACT

The present invention relates to dosing regimens and methods of administration that are useful for treating RSV infection in patients, such as pediatric patients. The methods described herein involve administration of compound (A)

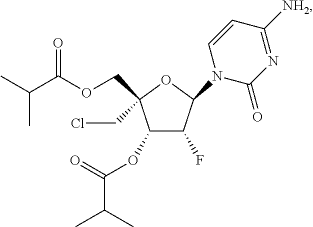 [SMILES: CC(C)C(=O)OC[C@@]1(CCl)O[C@@H](n2ccc(N)nc2=O)[C@H](F)[C@@H]1OC(=O)C(C)C]

or a pharmaceutically acceptable salt thereof.

This application claims priority to U.S. Provisional Patent Application No. 62/413,801 filed Oct. 27, 2016, which is incorporated herein in its entirety.

### FIELD

The present application relates to the fields of chemistry, biochemistry and medicine. More particularly, disclosed herein are methods of ameliorating and/or treating a Respiratory Syncytial Virus (RSV) infection.

### BACKGROUND

RSV, a species of pneumovirus, is a highly prevalent and contagious virus that often results in lower respiratory tract infections (LRTIs) which may cause other serious complications. RSV is the most common cause of bronchiolitis and pneumonia in children under one year of age in the world, and can be the cause of tracheobronchitis in older children and adults. RSV is the primary cause of hospitalization among infants with almost all experiencing their first RSV infection by the age of two. In developed countries, 1% to 3% of all children with RSV infection are hospitalized. In 2005, an estimated 33.4 million cases worldwide of acute lower respiratory infection (ALRI) due to the burden of RSV were reported with 3.4 million cases of hospital admissions. Furthermore, in the same period between 53,250 to 199,000 deaths as a result of RSV-associated ALRI was reported. According to the latest epidemiological study, the mortality from complications due to this disease stands at close to 118,200 cases annually. Moreover, the study reported a significant burden of RSV infection in neonates with an annual occurrence of nearly 40 episodes per 1,000 neonates. Such data reflect the significant impact of the disease on pediatric patients. No vaccines are currently approved for the prevention of RSV infection.

There are two subtypes of RSV, namely type A and type B, with differences in the envelope proteins on the viral shell. Risk factors associated with severity of the disease include, without limitation, young age, premature birth, passive smoke exposure, lack of breastfeeding, chronic lung disease, and congenital heart disease. Children often present with initial symptoms of cough, fever, and rhinorrhea which develop into wheezing, tachypnea, rales as well as heightened respiratory effort. Infants may also exhibit characteristics of decrease in appetite, vomiting, headache, sore throat, or irritability. Pediatric patients with underlying co-morbidities such as immune or chronic lung disease may progressively become worse.

RSV-related infections take a considerable toll, economically as well as physically. In 2000, close to 98% of all hospitalizations related to RSV infections in the U.S. were reported in children less than 5 years of age. In addition, hospitalizations due to RSV-related infections (USD $394 million) together with all other medical encounters (USD $258 million) for these children was an estimated USD $652 million of the total annual medical costs. The average cost of RSV-related hospitalization was an estimated USD $14,832 annually for all infants. Furthermore, the health care costs of RSV-related hospitalization in the U.S. for high-risk infants up to one year of age are reportedly from USD $20,160 to $39,399 annually. At the same time, the average costs of intensive care unit (ICU) admissions were an estimated USD $35,000 to $89,000 for RSV-related hospitalizations with higher costs for younger infants (less than 90 days old) compared to the older infants. A study of Japanese inpatients less than 5 years of age, diagnosed with RSV, reflected a mean total health care cost of USD $3,344 per hospitalization.

A challenge in the development of drugs for treatment and prophylaxis of RSV disease in pediatric patients is the limited body of knowledge around the pathogenesis of RSV infection. Underlying this challenge is an uncertainty in the role of RSV cytotoxicity versus that of the host immune response in RSV disease. Currently, two drugs are FDA approved for prevention or treatment of RSV LRTI in pediatric patients: palivizumab for prophylaxis and aerosolized ribavirin for treatment. Palivizumab (a monoclonal antibody) is approved for the prevention of serious lower respiratory tract disease caused by RSV in children at high risk of RSV disease but it has no therapeutic efficacy. The approval was based on results of a double-blind placebo controlled study of 1,502 patients 24 months of age or younger with bronchopulmonary dysplasia (BPD) or infants with premature birth who were 6 months of age or younger at study entry. Since most children affected with RSV infection are usually healthy prior to hospitalization, control strategies targeting only high-risk children will have a limited effect on decreasing the overall disease burden of RSV infections.

In regard to aerosolized ribavirin, the drug was approved by the FDA for treatment of hospitalized infants and young children with severe LRTIs caused by RSV. A meta-analysis by Randolph and Wang (1996) cited many methodological errors in the studies that had supported aerosolized ribavirin's clinical benefits, and the authors concluded that treatment with aerosolized ribavirin failed to impart any clinically significant benefits. At present, health care providers' perceptions of limited clinical benefits, in addition to concerns for mutagenicity, carcinogenicity, and teratogenicity with ribavirin, has resulted in infrequent use of ribavirin for the treatment of RSV-associated illness. Currently, ribavirin is used primarily when the outcome of an RSV LRTI could be fatal, such as in RSV infections in bone marrow transplant patients.

Given the limited options for treating or preventing RSV, supportive care is what remains to provide relief to pediatric patients suffering from RSV. Such supportive care include antibiotics, which are usually prescribed to treat bacterial infections; over-the-counter medication, which are not effective in treating RSV but may ameliorate some of the symptoms; oxygen therapy, mechanical ventilation, nutrition, and fluids. In severe cases, a nebulized bronchodilator, such as albuterol, may be prescribed to relieve some of the symptoms, such as wheezing. As such, there is a need for a drug and associated dosing regimens that treat RSV in pediatric patients, either in the outpatient setting (e.g. to reduce the severity of infection and prevent possible hospitalizations) or in the hospital setting (e.g. to ameliorate the severity of symptoms and duration of times spent in the hospital).

### SUMMARY OF THE INVENTION

The invention generally relates to dosing regimens and methods of administration for treating RSV infection in patients, wherein the methods involve administration of compound (A) (used throughout the specification as a designation for the following structure:

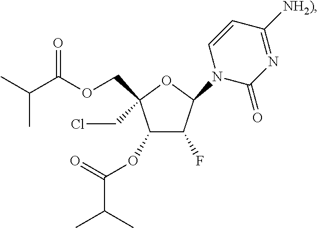

or a pharmaceutically acceptable salt thereof.
One aspect of the invention relates to methods of the invention involving administering, to a human pediatric patient in need of treatment of RSV, compound (A) or a pharmaceutically acceptable thereof according to a dosing regimen comprising a first dose of compound (A), or a pharmaceutically acceptable salt thereof, in the range of 30 mg/kg-70 mg/kg; and one or more second doses of compound (A), or a pharmaceutically acceptable salt thereof, in the range of 10 mg/kg-50 mg/kg. In some embodiments, the first dose of compound (A), or a pharmaceutically acceptable salt thereof, is in the range of 40 mg/kg-60 mg/kg and the one or more second doses is in the range of 20 mg/kg-40 mg/kg. In some instances, the first dose is 60 mg/kg while the second dose is selected to be 40 mg/kg. In other instances, the first dose is 40 mg/kg while the second dose is selected to be 20 mg/kg. In some embodiments, the first dose and the one or more second doses are administered BID. In certain embodiments, the dosing regimen comprises a single first dose and nine second doses of compound (A) or a pharmaceutically acceptable salt thereof. Compound (A) may be administered orally to the human pediatric patient, e.g. in a liquid formulation, e.g. a suspension. In further embodiments, second doses are administered BID. In other embodiments of the invention, the doses are administered QD. In still other embodiments, the dosing regimen spans a period of three days to seven days. In some of these embodiments, the dosing regimen spans a period of five to six days with the first dose being a loading dose and the second dose(s) being maintenance dose(s), administered BID. The dosing regimens described herein are employed for a human pediatric patient, wherein the pediatric patient is a child or an infant. In some embodiments of the invention, methods of treating RSV involve orally administering compound (A) or a pharmaceutically acceptable salt thereof to a human pediatric patient. Compound (A) or a pharmaceutically acceptable salt thereof can be administered in a liquid formulation, for example, a suspension. In some embodiments, the first dose is a loading dose of 30 mg/kg and each second dose is a maintenance dose of 6 mg/kg. In other embodiments, the first dose is a loading dose of 30 mg/kg and each second dose is a maintenance dose of 10 mg/kg. In still other embodiments, the first dose is a loading dose of 40 mg/kg and each second dose is a maintenance dose of 20 mg/kg. In still other embodiments, the first dose is a loading dose of 60 mg/kg and each second dose is a maintenance dose of 40 mg/kg. In some instances, a method of the invention is used to treat a child infected with RSV. In other instances, a method of the invention is used to treat an infant infected with RSV.

The invention also relates to methods of treating RSV, wherein the method comprises administering to a human pediatric patient infected with RSV, compound (A) or a pharmaceutically acceptable salt thereof, according to a dosing regimen comprising a first dose at 60 mg/kg followed by nine second doses at 40 mg/kg BID. In some embodiments, compound (A) or a pharmaceutically acceptable salt thereof is administered orally to the human pediatric patient, e.g. a child, an infant, a neonate. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

The invention also relates to methods of treating RSV, wherein the method comprises administering to a human pediatric patient infected with RSV, compound (A) or a pharmaceutically acceptable salt thereof, according to a dosing regimen comprising a first dose at 40 mg/kg followed by nine second doses at 20 mg/kg BID. In some embodiments, compound (A) or a pharmaceutically acceptable salt thereof is administered orally to the human pediatric patient, e.g. a child, an infant, a neonate. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

Another aspect of the invention also relates to methods of treating RSV, wherein the method comprises orally administering to a human pediatric patient infected with RSV, compound (A) or a pharmaceutically acceptable salt thereof, according to a dosing regimen of about 40 mg/kg to about 60 mg/kg BID, wherein the dosing regimen spans three to seven days. In certain embodiments, about 40 mg/kg to about 60 mg/kg of compound (A) or a pharmaceutically acceptable salt thereof is administered BID, with a total of ten doses administered over the course of the dosing regimen. In some embodiments, the dosing regimen spans a period of five days. In other embodiments, the dosing regimen spans a period of five to six days. In still other embodiments, methods of the invention involve administering to the human pediatric patient 60 mg/kg BID of compound (A) or a pharmaceutically acceptable salt thereof. In still further embodiments, methods of the invention involve administering to the human pediatric patient 40 mg/kg BID of compound (A) or a pharmaceutically acceptable salt thereof. In yet other embodiments, compound (A) or a pharmaceutically acceptable salt thereof is administered orally to the human pediatric patient, e.g. a child, an infant, a neonate. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

Yet another aspect of the invention relates to methods of treating RSV in a human pediatric patient, wherein the method comprises orally administering to the human pediatric patient infected with RSV, compound (A), or a pharmaceutically acceptable salt thereof, according to a dosing regimen comprising a loading dose followed by nine maintenance doses BID, wherein said loading dose and maintenance doses are administered at levels sufficient to effect a mean AUC₀₋₂₄ of compound (C):

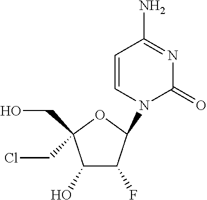 [SMILES: Nc1ccn([C@@H]2O[C@@](CO)(CCl)[C@@H](O)[C@H]2F)c(=O)n1]

in said human pediatric patient in the range of about 8,000 ng*hr/mL to about 20,000 ng*hr/mL following administration of compound (A) or the pharmaceutically acceptable salt thereof. In some embodiments of the invention, the loading dose and maintenance doses are administered at levels sufficient to effect a mean AUC₀₋₂₄ of compound (C) in said human pediatric patient in the range of about 12,000 ng*hr/mL to about 20,000 ng*hr/mL following administration of compound (A) or the pharmaceutically acceptable salt thereof. In other embodiments of the invention, the loading dose and maintenance doses are administered at levels sufficient to effect a mean AUC₀₋₂₄ of compound (C) in said human pediatric patient in the range of about 12,000 ng*hr/mL to about 19,000 ng*hr/mL following administration of compound (A) or the pharmaceutically acceptable salt thereof. In still other embodiments of the invention, the loading dose and maintenance doses are administered at levels sufficient to effect a mean AUC₀₋₂₄ of compound (C) in said human pediatric patient in the range of about 8,000 ng*hr/mL to about 12,000 ng*hr/mL following administration of compound (A) or the pharmaceutically acceptable salt thereof. In some of these embodiments, the loading dose is selected from the range of 40 mg/kg to 60 mg/kg and the maintenance dose is selected from the range of 20 mg/kg to 40 mg/kg. In certain embodiments, the loading dose is selected to be 40 mg/kg and the maintenance dose 20 mg/kg. Alternatively, the loading dose is selected to be 60 mg/kg and the maintenance dose 40 mg/kg. In methods of the present invention, compound (A) or a pharmaceutically acceptable salt thereof can be administered orally to the human pediatric patient, e.g. a child, an infant, a neonate. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

Some embodiments described herein generally relate to a method for ameliorating or treating a pneumovirus infection that can include administering a first dosage of compound (A), or a pharmaceutically acceptable salt thereof, and administering multiple separate second dosages of compound (A), or a pharmaceutically acceptable salt thereof, to a patient suffering from the pneumovirus infection; and wherein compound (A) is

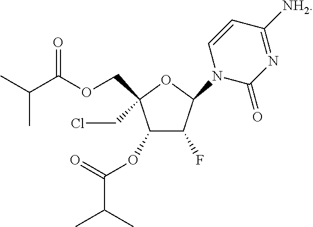 [SMILES: CC(C)C(=O)OC[C@@]1(CCl)O[C@@H](n2ccc(N)nc2=O)[C@H](F)[C@@H]1OC(=O)C(C)C]

Other embodiments described herein generally relate to a method for ameliorating or treating a pneumovirus infection that can include contacting a cell infected with the pneumovirus with an effective amount of a compound selected from compound (A) and compound (B), or a pharmaceutically acceptable salt of the foregoing; wherein the method can include administering compound (A), or a pharmaceutically acceptable salt thereof, in a first dosage and administering compound (A), or a pharmaceutically acceptable salt thereof, in multiple separate second dosages; and wherein compound (A) is

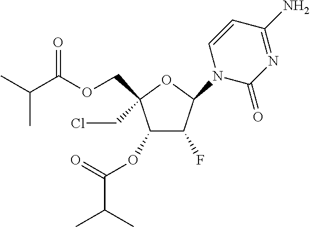 [SMILES: CC(C)C(=O)OC[C@@]1(CCl)O[C@@H](n2ccc(N)nc2=O)[C@H](F)[C@@H]1OC(=O)C(C)C]

and compound (B) is

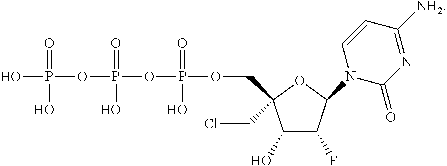 [SMILES: Nc1ccn([C@@H]2O[C@](CCl)(COP(=O)(O)OP(=O)(O)OP(=O)(O)O)[C@@H](O)[C@H]2F)c(=O)n1]

Still other embodiments described herein generally relate to a method for inhibiting the replication of a pneumovirus that can include contacting a cell infected with the pneumovirus with an effective amount of a compound selected from compound (A) and compound (B), or a pharmaceutically acceptable salt of the foregoing; wherein the method can include administering compound (A), or a pharmaceutically acceptable salt thereof, in a first dosage and administering compound (A), or a pharmaceutically acceptable salt thereof, in multiple separate second dosages; and wherein compound (A) is

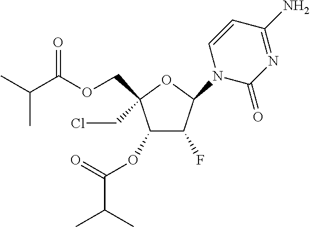 [SMILES: CC(C)C(=O)OC[C@@]1(CCl)O[C@@H](n2ccc(N)nc2=O)[C@H](F)[C@@H]1OC(=O)C(C)C]

and compound (B) is

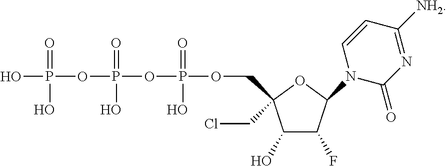 [SMILES: Nc1ccn([C@@H]2O[C@](CCl)(COP(=O)(O)OP(=O)(O)OP(=O)(O)O)[C@@H](O)[C@H]2F)c(=O)n1]

Some embodiments described herein generally relate to a method for ameliorating or treating a pneumovirus infection that can include administering a single dosage of compound (A), or a pharmaceutically acceptable salt thereof. Other embodiments described herein generally relate to a method for ameliorating or treating a pneumovirus infection that can include contacting a cell infected with the pneumovirus with an effective amount of a compound selected from compound (A) and compound (B), or a pharmaceutically acceptable salt of the foregoing, wherein the effective amount of a compound (A) and/or compound (B) or a pharmaceutically acceptable salt of the foregoing, is provided in a single dosage. Still other embodiments described herein generally relate to a method for inhibiting the replication of a pneumovirus that can include contacting a cell infected with the pneumovirus with an effective amount of a compound selected from compound (A) and compound (B), or a pharmaceutically acceptable salt of the foregoing, wherein the effective amount of a compound (A) and/or compound (B) or a pharmaceutically acceptable salt of the foregoing, is provided in a single dosage. In some embodiments, the pneumovirus is a respiratory syncytial virus (RSV).

### BRIEF DESCRIPTION OF THE DRAWINGS

FIG. 1 shows the change in RSV viral load following administration of Compound (A) or placebo for 5 Days in ITT-I population.

FIG. 2 provides the single and multiple doses of compound (A) used in a randomized, double-blind placebo controlled study in pediatric patients.

FIG. 3 provides the plasma exposure (AUC₀₋₂₄) of compound (C) following oral doses of 40/20 mg/kg or 60/40 mg/kg Compound (A) in pediatric patients, ages ranging from 1 to 12 months of age. The blue stars indicate actual compound (C) concentration in pediatric patients. Pharmacokinetic modeling was used to calculate AUC₀₋₂₄.

FIG. 4 is a schematic of the Phase 2, randomized, double-blind, placebo-controlled study of hospitalized infants and children referred to in Example 5.

### DETAILED DESCRIPTION

Lumicitabine, also referred to herein as Compound (A), is a first-in-class oral nucleoside analog for treatment of RSV in humans, including pediatric patients. When administered in vivo, lumicitabine (a prodrug) is converted to a nucleoside of the structure:

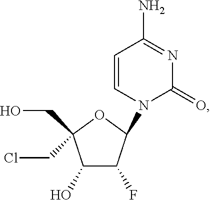 [SMILES: Nc1ccn([C@@H]2O[C@@](CO)(CCl)[C@@H](O)[C@H]2F)c(=O)n1]

also referenced herein as compound (C), the triphosphate of which is a selective inhibitor of RSV RNA polymerase activity.

As those of skill in the art would appreciate, efficacy of an antiviral drug is generally evaluated initially in adults then extrapolated to the pediatric population if the pathophysiology of the disease is similar in adults and pediatric patients. In the case of RSV however, the pathophysiology of the disease differs significantly between adult and pediatric patients. Not to be bound by theory, the difference can be attributed to key physiological and anatomical differences between the respiratory tracts of pediatric patients and adult patients; pediatric patients, especially infants, have smaller airways, which are more susceptible to compromise from inflammation caused by RSV infection. Therefore, extrapolation of efficacy data from adults to pediatric patients is challenging for various types of RSV LRTI in young children.

There are numerous challenges with establishing dosing regimens of lumicitabine for therapeutic use. Too low a dose would be inadequate for treating the RSV infection and exert pressure and selection of predominantly resistant viral populations. Too high a dose could raise incidence of adverse effects and toxicity. The inventors have discovered, after extensive analysis of clinical data, certain dosing regimens and methods of administering lumicitabine (compound (A), or a pharmaceutically acceptable salt thereof, which are suitable for RSV therapeutic use in pediatric patients. Moreover, the inventors have discovered that the exposure range over which lumicitabine appears to have clinically meaningful antiviral activity with acceptable safety is a mean AUC₀₋₂₄ of compound (C) of approximately 8,000-20,000 ng*hr/mL. Based on preliminary PK data in ongoing clinical study, pediatric lumicitabine doses equivalent to a first loading dose from about 30-70 mg/kg followed by nine maintenance doses at about 10-50 mg/kg BID achieve the targeted antiviral compound (C) plasma exposure in patients.

The invention also relates to methods of the invention involving administering, to a human pediatric patient in need of treatment of RSV, compound (A) or a pharmaceutically acceptable thereof according to a dosing regimen comprising a first dose of compound (A), or a pharmaceutically acceptable salt thereof, in the range of 30 mg/kg-70 mg/kg; and one or more second doses of compound (A), or a pharmaceutically acceptable salt thereof, in the range of 10 mg/kg-50 mg/kg. In some embodiments, the first dose of compound (A), or a pharmaceutically acceptable salt thereof, is in the range of 40 mg/kg-60 mg/kg and the one or more second doses is in the range of 20 mg/kg-40 mg/kg. In some instances, the first dose is 60 mg/kg while the second dose is selected to be 40 mg/kg. In other instances, the first dose is 40 mg/kg while the second dose is selected to be 20 mg/kg. In some embodiments, the first dose and the one or more second doses are administered BID. In certain embodiments, the dosing regimen comprises a single first dose and nine second doses of compound (A) or a pharmaceutically acceptable salt thereof. Compound (A) may be administered orally to the human pediatric patient, e.g. in a liquid formulation, e.g. a suspension. In further embodiments, second doses are administered BID. In other embodiments of the invention, the doses are administered QD. In still other embodiments, the dosing regimen spans a period of three days to seven days. In some of these embodiments, the dosing regimen spans a period of five to six days with the first dose being a loading dose and the second dose(s) being maintenance dose(s), administered BID. The dosing regimens described herein are employed for a human pediatric patient, wherein the pediatric patient is a child or an infant. In some embodiments of the invention, methods of treating RSV involve orally administering compound (A) or a pharmaceutically acceptable salt thereof to a human pediatric patient. Compound (A) or a pharmaceutically acceptable salt thereof can be administered in a liquid formulation, for example, a suspension. In some embodiments, the first dose is a loading dose of 30 mg/kg and each second dose is a maintenance dose of 6 mg/kg. In other embodiments, the first dose is a loading dose of 30 mg/kg and each second dose is a maintenance dose of 10 mg/kg. In still other embodiments, the first dose is a loading dose of 40 mg/kg and each second dose is a maintenance dose of 20 mg/kg. In still other embodiments, the first dose is a loading dose of 60 mg/kg and each second dose is a maintenance dose of 40 mg/kg. In some instances, a method of the invention is used to treat a child infected with RSV. In other instances, a method of the invention is used to treat an infant infected with RSV.

The invention also relates to methods of treating RSV, wherein the method comprises administering to a human pediatric patient infected with RSV, compound (A) or a pharmaceutically acceptable salt thereof, according to a dosing regimen comprising a first dose at 60 mg/kg followed by nine second doses at 40 mg/kg BID. In some embodiments, compound (A) or a pharmaceutically acceptable salt thereof is administered orally to the human pediatric patient, e.g. a child, an infant, a neonate. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

The invention also relates to methods of treating RSV, wherein the method comprises administering to a human pediatric patient infected with RSV, compound (A) or a pharmaceutically acceptable salt thereof, according to a dosing regimen comprising a first dose at 40 mg/kg followed by nine second doses at 20 mg/kg BID. In some embodiments, compound (A) or a pharmaceutically acceptable salt thereof is administered orally to the human pediatric patient, e.g. a child, an infant, a neonate. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

The invention also relates to methods of treating RSV, wherein the method comprises orally administering to a human pediatric patient infected with RSV, compound (A) or a pharmaceutically acceptable salt thereof, according to a dosing regimen of about 40 mg/kg to about 60 mg/kg BID, wherein the dosing regimen spans three to seven days. In certain embodiments, about 40 mg/kg to about 60 mg/kg of compound (A) or a pharmaceutically acceptable salt thereof is administered BID. In some embodiments, the dosing regimen spans a period of five days. In other embodiments, the dosing regimen spans a period of five to six days. In still other embodiments, methods of the invention involve administering to the human pediatric patient 60 mg/kg BID of compound (A) or a pharmaceutically acceptable salt thereof. In still further embodiments, methods of the invention involve administering to the human pediatric patient 40 mg/kg BID of compound (A) or a pharmaceutically acceptable salt thereof. In yet other embodiments, compound (A) or a pharmaceutically acceptable salt thereof is administered orally to the human pediatric patient, e.g. a child, an infant, a neonate. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

Yet another aspect of the invention relates to methods of treating RSV in a human pediatric patient, wherein the method comprises orally administering to the human pediatric patient infected with RSV, compound (A), or a pharmaceutically acceptable salt thereof, according to a dosing regimen comprising a loading dose followed by nine maintenance doses BID, wherein said loading dose and maintenance doses are administered at levels sufficient to effect a mean AUC₀₋₂₄ of compound (C):

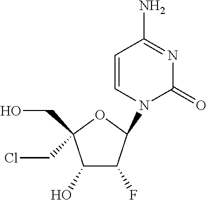 [SMILES: Nc1ccn([C@@H]2O[C@@](CO)(CCl)[C@@H](O)[C@H]2F)c(=O)n1]

in said human pediatric patient in the ange of about 8,000 ng*hr/mL to about 20,000 ng*hr/mL following administration of compound (A) or the pharmaceutically acceptable salt thereof. In some embodiments of the invention, the loading dose and maintenance doses are administered at levels sufficient to effect a mean AUC₀₋₂₄ of compound (C) in said human pediatric patient in the range of about 12,000 ng*hr/mL to about 20,000 ng*hr/mL following administration of compound (A) or the pharmaceutically acceptable salt thereof. In other embodiments of the invention, the loading dose and maintenance doses are administered at levels sufficient to effect a mean AUC₀₋₂₄ of compound (C) in said human pediatric patient in the range of about 12,000 ng*hr/mL to about 19,000 ng*hr/mL following administration of compound (A) or the pharmaceutically acceptable salt thereof. In still other embodiments of the invention, the loading dose and maintenance doses are administered at levels sufficient to effect a mean AUC₀₋₂₄ of compound (C) in said human pediatric patient in the range of about 8,000 ng*hr/mL to about 12,000 ng*hr/mL following administration of compound (A) or the pharmaceutically acceptable salt thereof. In some of these embodiments, the loading dose is selected from the range of 40 mg/kg to 60 mg/kg and the maintenance dose is selected from the range of 20 mg/kg to 40 mg/kg. In certain embodiments, the loading dose is selected to be 40 mg/kg and the maintenance dose 20 mg/kg. Alternatively, the loading dose is selected to be 60 mg/kg and the maintenance dose 40 mg/kg. In methods of the present invention, compound (A) or a pharmaceutically acceptable salt thereof can be administered orally to the human pediatric patient, e.g. a child, an infant, a neonate. Other loading and maintenance dose combinations may be used to target or exceed the antiviral plasma exposure ranges by using pharmacokinetic modeling that use both pediatric and adult human plasma pharmacokinetic data to estimate plasma exposures for a given lumicitabine dose. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

Some embodiments described herein relate to a method for ameliorating or treating a pneumovirus infection that can include administering a first dosage of compound (A), or a pharmaceutically acceptable salt thereof, and administering multiple separate second dosages of compound (A), or a pharmaceutically acceptable salt thereof, to a patient suffering from the pneumovirus infection. Other embodiments described herein relate to using a first dosage compound (A), or a pharmaceutically acceptable salt thereof, and multiple separate second dosages of compound (A), or a pharmaceutically acceptable salt thereof, in the manufacture of a medicament for ameliorating and/or treating a pneumovirus infection in a patient suffering from the pneumovirus infection. Still other embodiments described herein relate to a first dosage of compound (A), or a pharmaceutically acceptable salt thereof, and multiple separate second dosages of compound (A), or a pharmaceutically acceptable salt thereof, that can be used for ameliorating and/or treating a pneumovirus infection in a patient suffering from the pneumovirus infection.

Some embodiments disclosed herein relate to a method of ameliorating and/or treating a pneumovirus infection that can include contacting a cell infected with the pneumovirus with an effective amount of a compound selected from compound (A) and compound (B), or a pharmaceutically acceptable salt of the foregoing; wherein the method can include administering compound (A), or a pharmaceutically acceptable salt thereof, in a first dosage and administering compound (A), or a pharmaceutically acceptable salt thereof, in multiple separate second dosages. Other embodiments described herein relate to using a compound selected from compound (A) and compound (B), or a pharmaceutically acceptable salt of the foregoing, in the manufacture of a medicament for ameliorating and/or treating a pneumovirus infection that can include contacting a cell infected with the pneumovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages. Still other embodiments described herein relate to a compound selected from compound (A) and compound (B), or a pharmaceutically acceptable salt of the foregoing, that can be used for ameliorating and/or treating a pneumovirus infection by contacting a cell infected with the pneumovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages.

Some embodiments disclosed herein relate to a method of inhibiting replication of a pneumovirus that can include contacting a cell infected with the pneumovirus with an effective amount of compound (A) and/or compound (B), or a pharmaceutically acceptable salt of the foregoing; and wherein the method can include administering compound (A), or a pharmaceutically acceptable salt thereof, in a first dosage and administering compound (A), or a pharmaceutically acceptable salt thereof, in multiple separate second dosages. Other embodiments described herein relate to using compound (A) and/or compound (B), or a pharmaceutically acceptable salt of the foregoing, in the manufacture of a medicament for inhibiting replication of a pneumovirus that can include contacting a cell infected with the pneumovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages. Still other embodiments described herein relate to compound (A) and/or compound (B), or a pharmaceutically acceptable salt of the foregoing, that can be used for inhibiting replication of a pneumovirus by contacting a cell infected with the pneumovirus with an effective amount of compound and/or compounds; and wherein the use can include administering compound (A), or a pharmaceutically acceptable salt thereof, in a first dosage and administering compound (A), or a pharmaceutically acceptable salt thereof, in multiple separate second dosages.

Some embodiments described herein relate to a method of inhibiting a pneumovirus polymerase can include contacting a cell infected with a pneumovirus with an effective amount of compound (A) and/or compound (B), or a pharmaceutically acceptable salt of the foregoing; and wherein the method can include administering compound (A), or a pharmaceutically acceptable salt thereof, in a first dosage and administering compound (A), or a pharmaceutically acceptable salt thereof, in multiple separate second dosages. Other embodiments described herein relate to using compound (A) and/or compound (B), or a pharmaceutically acceptable salt of the foregoing, in the manufacture of a medicament for inhibiting a pneumovirus polymerase that can include contacting a cell infected with the pneumovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages. Still other embodiments described herein relate to compound (A) and/or compound (B), or a pharmaceutically acceptable salt of the foregoing, that can be used for inhibiting a pneumovirus polymerase that can include contacting a cell infected with the pneumovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages.

Some embodiments described herein relate to a method of ameliorating and/or treating a respiratory infection (for example, an upper and/or lower respiratory infection) in a patient suffering from the respiratory infection, wherein the respiratory infection is caused by a pneumovirus infection, that can include administering a first dosage of compound (A), or a pharmaceutically acceptable salt thereof, and administering multiple separate second dosages of compound (A), or a pharmaceutically acceptable salt thereof. Other embodiments described herein relate to a method of ameliorating and/or treating a respiratory infection in a patient suffering from the respiratory infection, wherein the respiratory infection is caused by a pneumovirus infection, that can include contacting a cell infected with pneumovirus in the patient with an effective amount of compound (A) and/or compound (B), or a pharmaceutically acceptable salt of the foregoing; and wherein the method can include administering compound (A), or a pharmaceutically acceptable salt thereof, in a first dosage and administering compound (A), or a pharmaceutically acceptable salt thereof, in multiple separate second dosages. Still other embodiments described herein relate to using compound (A) and/or compound (B), or a pharmaceutically acceptable salt of the foregoing, in the manufacture of a medicament for ameliorating and/or treating a respiratory infection, wherein the respiratory infection is due to a pneumovirus infection, that can include administering a first dosage of compound (A), or a pharmaceutically acceptable salt thereof, and administering multiple separate second dosages of compound (A), or a pharmaceutically acceptable salt thereof. Yet still other embodiments described herein relate to compound (A) and/or compound (B), or a pharmaceutically acceptable salt of the foregoing, that can be used for ameliorating and/or treating a respiratory infection in a patient suffering from the respiratory infection, wherein the respiratory infection is from a pneumovirus infection, that can include contacting a cell infected with the pneumovirus in the patient with an effective amount of compound (A) and/or compound (B), or a pharmaceutically acceptable salt of the foregoing; and wherein the use can include administering compound (A), or a pharmaceutically acceptable salt thereof, in a first dosage and administering compound (A), or a pharmaceutically acceptable salt thereof, in multiple separate second dosages. Some embodiments described herein relate to compound (A) and/or compound (B), or a pharmaceutically acceptable salt of the foregoing, that can be used for ameliorating and/or treating a respiratory infection in a patient suffering from the respiratory infection, wherein the respiratory infection is from a pneumovirus infection, that can include administering a first dosage of compound (A), or a pharmaceutically acceptable salt thereof, and administering multiple separate second dosages of compound (A), or a pharmaceutically acceptable salt thereof. Other embodiments described herein relate to compound (A) and/or compound (B), or a pharmaceutically acceptable salt of the foregoing, that can be used for ameliorating and/or treating a respiratory infection in a patient suffering from the respiratory infection, wherein the respiratory infection is from a pneumovirus infection, that can include contacting a cell infected with the pneumovirus in the patient with an effective amount of compound (A) and/or compound (B), or a pharmaceutically acceptable salt of the foregoing; and wherein the use can include administering compound (A), or a pharmaceutically acceptable salt thereof, in a first dosage and administering compound (A), or a pharmaceutically acceptable salt thereof, in multiple separate second dosages. Examples of respiratory infections include those described herein, such as, colds, croup, pneumonia, bronchitis, tracheobronchitis and bronchiolitis. A non-limiting list of symptoms of a respiratory infection can include a cough, runny nose, nasal congestion, sore throat, fever, difficulty breathing, abnormally rapid breathing, wheezing vomiting, diarrhea and ear infections.

In some embodiments, a method and/or use described herein can be used to ameliorate and/or treat a RSV infection, a respiratory infection attributable to a RSV infection and/or one or more symptoms of a RSV infection. A compound described herein may be active against more than one type of RSV. In some embodiments, a method and/or use described herein can be used to ameliorate and/or treat an infection caused by RSV type A. In other embodiments, a method and/or use described herein can be used to ameliorate and/or treat an infection caused by RSV type B. In still other embodiments, a method and/or use described herein can be used to ameliorate and/or treat an infection caused by RSV types A and B.

The compounds (A) and (B), or a pharmaceutically acceptable salt of the foregoing, are described in U.S. Publication Nos. 2013/0165400 and 2015/0051167 and International Publication Nos. WO 2013/142525 and WO 2013/142525, all of which are hereby incorporated by reference in their entireties. Those skilled in the art understand that once compound (A), or a pharmaceutically acceptable salt thereof, is absorbed, the groups attached to 3′ and 5′ positions can be easily removed by esterases, proteases and/or other enzymes. Once inside the cell, the triphosphate (compound (B), or a pharmaceutically acceptable salt thereof) can be formed via metabolization by cellular enzymes. Compound (B), or a pharmaceutically acceptable salt thereof, inhibits RNA polymerase activity via a chain termination mechanism, and has a half-life of approximately 17.6 hours.

In the context of treating pediatric patients infected with RSV, the first dose of compound (A), or a pharmaceutically salt thereof, can include an amount of compound (A), or a pharmaceutically salt thereof, in the range of 30 mg/kg to 70 mg/kg. Alternatively, the first dose may be selected from the range of 40 mg/kg to 60 mg/kg. Still alternatively, the first dose may be selected from the range of 30 mg/kg to 50 mg/kg. Still alternatively, the first dose may be selected from the range of 50 mg/kg to 70 mg/kg. In certain embodiments, the first dose of compound (A), or a pharmaceutically acceptable salt thereof, is selected from the group consisting of 30 mg/kg, 35 mg/kg, 40 mg/kg, 45 mg/kg, 50 mg/kg, 55 mg/kg, 60 mg/kg, 65 mg/kg or 70 mg/kg.

In the context of treating pediatric patients infected with RSV, the second dose of compound (A), or a pharmaceutically salt thereof, can include an amount of compound (A), or a pharmaceutically salt thereof, in the range of 10 mg/kg to 50 mg/kg. Alternatively, the second dose may be selected from the range of 20 mg/kg to 40 mg/kg. Still alternatively, the second dose may be selected from the range of 30 mg/kg to 50 mg/kg. Still alternatively, the second dose may be selected from the range of 10 mg/kg to 30 mg/kg. In certain embodiments, the second dose of compound (A), or a pharmaceutically acceptable salt thereof, is selected from the group consisting of 10 mg/kg, 15 mg/kg, 20 mg/kg, 25 mg/kg, 30 mg/kg, 35 mg/kg, 40 mg/kg, 45 mg/kg or 50 mg/kg.

As used herein, the term “approximately” in relation to a numerical value (“x”) means x±8%.

As used herein, BID or twice per day means twice in any period of a 24 hour period; QD or once per day means once in any period of a 24 hour period.

In some embodiments, different amounts of compound (A), or a pharmaceutically acceptable salt thereof, can be given during treatment. In other embodiments, the same amounts of compound (A), or a pharmaceutically acceptable salt thereof, can be given during treatment. In some embodiments, one or more “loading” dosages that can include an amount(s) of compound (A), or a pharmaceutically acceptable salt thereof, can be given followed by several “maintenance” dosages that can include an amount(s) of compound (A), or a pharmaceutically acceptable salt thereof. The terms “loading dosage” and “maintenance dosage” are used herein as understood by those skilled in the art. A “loading dosage” is an amount of a compound provided for the purpose of establishing a therapeutic level of the compound in the target tissue (for example, the lung). A “maintenance dosage” is an amount of a compound provided to maintain a desired level of the compound in the target tissue (such as the lung). In some embodiments, the amount of the loading dosage can be greater than the amount of each maintenance dosage. In other embodiments, the amount of the loading dosage can be the same as the amount of each maintenance dosage. In some embodiments, the amount of compound being maintained is the active metabolite in the target tissue (for example, an amount of compound (B), or a pharmaceutically acceptable salt thereof, in lung tissue). Those skilled in the art understand that the loading dosage that may include a single dosage or multiple dosages is given for a first period of time followed by one or more maintenance dosages for a second period of time. As those of skill in the art would appreciate, the loading and maintenance doses can respectively be adjusted so that the plasma concentrations (Cmax) and/or the plasma area under the curve (AUC) are maintained within a given range.

As described herein, multiple second doses of compound (A), or a pharmaceutically acceptable salt thereof, can be provided. In some embodiments, the number of second dosages can be in the range of 2 to 20 separate second doses of compound (A), or a pharmaceutically acceptable salt thereof. In other embodiments, the number of second doses can be in the range of 2 to 15 separate second doses of compound (A), or a pharmaceutically acceptable salt thereof. In still other embodiments, the number of second doses can be in the range of 2 to 12 separate second doses of compound (A), or a pharmaceutically acceptable salt thereof. In still other embodiments, the number of second doses can be in the range of 2 to 10 separate second doses of compound (A), or a pharmaceutically acceptable salt thereof. In yet other embodiments, the dosing regimen consists of nine second doses of compound (A) or a pharmaceutically acceptable salt thereof. In some embodiments, the number of second doses can be more than 2 separate second doses of compound (A), or a pharmaceutically acceptable salt thereof. In other embodiments, the number of second doses can be more than 5 separate second doses of compound (A), or a pharmaceutically acceptable salt thereof. In still other embodiments, the number of second doses can be more than 8 separate second doses of compound (A), or a pharmaceutically acceptable salt thereof.

The frequency and length of administration of compound (A), or a pharmaceutically salt thereof, can vary. In some embodiments, compound (A), or a pharmaceutically salt thereof, can be dosed once daily. In other embodiments, compound (A), or a pharmaceutically salt thereof, can be dosed twice daily. For example, compound (A), or a pharmaceutically salt thereof, can be provided at a first time period and then at a second time period, wherein the first time period and the second time period are separated by at least 8 hours. In some embodiments, the first dosage of compound (A), or a pharmaceutically acceptable salt thereof, can be given in a single dosage once daily. In other embodiments, the first dosage of compound (A), or a pharmaceutically acceptable salt thereof, can be given in two doses at different times. As an example, one of the first doses can be given at a first time period and other of the first doses can be given at a second time period, wherein the two time periods are separated by one or more hours (for example, separated by 8-14 hours). In some embodiments, the two doses of the first dosage are separated by approximately 12 hours.

The initial second dosage and subsequent second doses can be administered at various times. In some embodiments, the initial second dosage can be provided in the range of 8 hours to 14 hours after completion of the first dosage (such as after the final dosage of the first dosage). In other embodiments, the initial second dosage can be provided in the range of 8 hours to 18 hours after completion of the first dosage (such as after the final dosage of the first dosage). In some embodiments, the initial second dosage can be provided approximately 12 hours after completion of the first dosage. In some embodiments, the first maintenance dosage can be provided in the range of 8 hours to 18 hours after completion of the last loading dosage. In some embodiments, the first maintenance dosage can be provided approximately 12 hours after completion of the last loading dosage. The subsequent second doses can be provided at approximate regular intervals following the initial second dosage. As an example, each subsequent second dosage can be given in approximate 8 hours to 14 hours intervals. In some embodiments, subsequent second doses can be provided approximately every 12 hours after the initial second dosage. In some embodiments, each subsequent maintenance dosage can be provided approximately every 12 hours after the first maintenance dosage. In some embodiments, each second dosage of compound (A), or a pharmaceutically acceptable salt thereof, can be given once daily. In other embodiments, each second dosage of compound (A), or a pharmaceutically acceptable salt thereof, can be given twice daily. One example of dosing is the first dosage of compound (A), or a pharmaceutically acceptable salt thereof, can be given once daily, and each second dosage of compound (A), or a pharmaceutically acceptable salt thereof, can be given twice daily. In some embodiments, the loading dosage of compound (A), or a pharmaceutically acceptable salt thereof, can be given once daily. In other embodiments, loading dosage of compound (A), or a pharmaceutically acceptable salt thereof, can be given twice daily. In some embodiments, each maintenance dosage of compound (A), or a pharmaceutically acceptable salt thereof, can be given once daily. In other embodiments, each maintenance dosage of compound (A), or a pharmaceutically acceptable salt thereof, can be given twice daily. In some embodiments, the first dosage and each second dosage can be administered sequentially.

In some embodiments, compound (A), or a pharmaceutically acceptable salt thereof, can be provided for a total number of at least 3 days. In other embodiments, compound (A), or a pharmaceutically acceptable salt thereof, can be provided for a total number of at least 5 days. In still other embodiments, compound (A), or a pharmaceutically acceptable salt thereof, can be provided for a total number of at least 7 days. In yet still other embodiments, compound (A), or a pharmaceutically acceptable salt thereof, can be provided for a total number of at least 14 days. In some embodiments, compound (A), or a pharmaceutically acceptable salt thereof, can be provided for a total number of at least 28 days. In some embodiments, compound (A), or a pharmaceutically acceptable salt thereof, can be provided for a total time period in the range of 3 days to 14 days. In other embodiments, compound (A), or a pharmaceutically acceptable salt thereof, can be provided for a total time period in the range of 3 days to 30 days. In still other embodiments, compound (A), or a pharmaceutically acceptable salt thereof, can be provided for a total time period in the range of 4 days to 6 days. In still other embodiments, compound (A), or a pharmaceutically acceptable salt thereof, can be provided for a total time period in the range of 3 days to 10 days.

In some embodiments, compound (A), or a pharmaceutically acceptable salt thereof, can be provided for a time period that can be ≥1 day and ≤7 days. In other embodiments, compound (A), or a pharmaceutically acceptable salt thereof, can be provided for a time period that can be ≥1 day and ≤6 days. In still other embodiments, compound (A), or a pharmaceutically acceptable salt thereof, can be provided for a time period that can be ≥1 day and ≤5 days. In some embodiments, compound (A), or a pharmaceutically acceptable salt thereof, can be provided for a time period of 3 days. In other embodiments, compound (A), or a pharmaceutically acceptable salt thereof, can be provided for a time period of 4 days. In still other embodiments, compound (A), or a pharmaceutically acceptable salt thereof, can be provided for a time period of 5 days. In yet still other embodiments, compound (A), or a pharmaceutically acceptable salt thereof, can be provided for a time period of 6 days. In some embodiments, compound (A), or a pharmaceutically acceptable salt thereof, can be provided for a time period of 7 days. In some embodiments, compound (A), or a pharmaceutically acceptable salt thereof, can be provided for a time period in the range of 4 days to 6 days. In other embodiments, compound (A), or a pharmaceutically acceptable salt thereof, can be provided for a time period in the range of 3 days to 7 days. In still other embodiments, compound (A), or a pharmaceutically acceptable salt thereof, can be provided for a time period in the range of 3 days to 10 days. In some embodiments, compound (A), or a pharmaceutically acceptable salt thereof, can be provided over a consecutive number of days (such as 3, 4, 5, 6, 7, 8, 9 and/or 10 consecutive days).

In some embodiments, the first dosage of compound (A), or a pharmaceutically acceptable salt thereof, can be given in a first time period (such as immediately after or within the first 12-24 hours following a positive diagnosis of a RSV infection) followed by several second doses of compound (A), or a pharmaceutically acceptable salt thereof, for a second time period (for example, multiple days). In some embodiments, the second doses of compound (A), or a pharmaceutically acceptable salt thereof, can be given for at least 3 days. In other embodiments, the second doses of compound (A), or a pharmaceutically acceptable salt thereof, can be given for at least 4 days. In some embodiments, the second doses of compound (A), or a pharmaceutically acceptable salt thereof, can be given for a number of days in the range of 3 to 7 days. In other embodiments, the second doses of compound (A), or a pharmaceutically acceptable salt thereof, can be given for a number of days in the range of 3 to 14 days. In still other embodiments, the second doses of compound (A), or a pharmaceutically acceptable salt thereof, can be given for a number of days in the range of 3 to 30 days. In some embodiments, the second doses of compound (A), or a pharmaceutically acceptable salt thereof, can be provided for a number of days in the range of 4 days to 6 days.

In some embodiments of the invention, the method of treating RSV in pediatric patients comprises orally administering a single dose which is selected from the range from about 30 mg/kg to about 70 mg/kg BID, wherein the dosing regimen spans three to seven days. For instance, the dose of compound (A), or a pharmaceutically acceptable salt thereof, administered can be 30 mg/kg, 35 mg/kg, 40 mg/kg, 45 mg/kg, 50 mg/kg, 55 mg/kg, 60 mg/kg, 65 mg/kg or 70 mg/kg. In certain embodiments, about 40 mg/kg to about 60 mg/kg of compound (A) or a pharmaceutically acceptable salt thereof is administered BID, with a total of ten doses administered over the course of the dosing regimen. In some embodiments, the dosing regimen spans a period of five days. In other embodiments, the dosing regimen spans a period of five to six days. In still other embodiments, methods of the invention involve administering to the human pediatric patient 60 mg/kg BID of compound (A) or a pharmaceutically acceptable salt thereof. In still further embodiments, methods of the invention involve administering to the human pediatric patient 40 mg/kg BID of compound (A) or a pharmaceutically acceptable salt thereof. In yet other embodiments, compound (A) or a pharmaceutically acceptable salt thereof is administered orally to the human pediatric patient, e.g. a child, an infant, a neonate. In other embodiments of the invention, compound (A) or a pharmaceutically acceptable thereof is administered in a liquid formulation, e.g. a suspension. In still other embodiments of the invention, the pediatric patient is infected with RSV type A. In still other embodiments of the invention, the pediatric patient is infected with RSV type B.

Examples of regimens that include some of the embodiments described herein are provided in Tables 1, 2 and 3. The amounts in Tables 1 and 2 are for compound (A), or a pharmaceutically acceptable salt thereof, for use in adults. The amounts in Table 3 are for compound (A), or a pharmaceutically acceptable salt thereof, for use in children and infants.

TABLE 1

| Example | | No. of First | Each Second | Frequency of |
|---|---|---|---|---|
| Regimen | First Dosage | Dosages | Dosage | Second Dosages |
| 1 | 700 mg-800 mg | 1 | 100 mg-200 mg | 8-14 hour |
| | | | | intervals |
| 2 | 700 mg-800 mg | 1 | 450 mg-550 mg | 8-14 hour |
| | | | | intervals |
| 3 | 700 mg-800 mg | 1 | 325 mg-425 mg | 8-14 hour |
| | | | | intervals |
| 4 | 1400 mg-1600 mg | 2 | 100 mg-200 mg | 8-14 hour |
| | | each dosage | | intervals |
| | | 700 mg-800 mg | | |
| 5 | 1400 mg-1600 mg | 2 | 450 mg-550 mg | 8-14 hour |
| | | each dosage | | intervals |
| | | 700 mg-800 mg* | | |
| 6 | 1400 mg-1600 mg | 2 | 325 mg-425 mg | 8-14 hour |
| | | each dosage | | intervals |
| | | 700 mg-800 mg* | | |

*The two dosages are provided at different times (e.g., 1 dose in the morning and 1 dose at night).

TABLE 2

| Example | Dose Regimen | | | |
|---|---|---|---|---|
| Regimen | (mg) | Dose 1 | Dose 2 | Doses 3-10 |
| 7 | 100 (BID) | 100 mg (BID) | | |
| 8 | 100 (QD) | 100 mg (QD) | | |
| 9 | 325/80 | 325 mg | 80 mg | 80 mg (BID) |
| 10 | 400/100 | 400 mg | 100 mg | 100 mg (BID) |
| 11 | 200/200/100 | 200 mg | 200 mg | 100 mg (BID) |
| 12 | 750/750/500 | 750 mg | 750 mg | 500 mg (BID) |
| 13 | 750/500 | 750 mg | 500 mg | 500 mg (BID) |
| 14 | 200/50 | 200 mg | 50 mg | 50 mg (BID) |
| 15 | 400/50 | 400 mg | 50 mg | 50 mg (BID) |
| 16 | 400/0/50 | 400 mg | 0 mg | 50 mg (BID) |
| 17 | 50 (BID) | 50 mg (BID) | | |
| 18 | 200 (QD) | 200 mg (QD) | | |
| 19 | 400 (QD) | 400 mg (QD) | | |
| 20 | 500 (BID) | 500 mg (BID) | | |
| 21 | 100 q6h (BID) | 100 mg q6h | | 100 mg (BID) |
| | | (BID) | | |
| 22 | 200 q6h (BID) | 200 mg q6h | | 200 mg (BID) |
| | | (BID) | | |
| 23 | 400 q6h (BID) | 400 mg q6h | | 400 mg (BID) |
| | | (BID) | | |
| 24 | 750/150 | 750 mg | 150 mg | 150 mg (BID) |

TABLE 3

| Example | Dose Regimen | | | | |
|---|---|---|---|---|---|
| Regimen | (mg) | | Dose 1 | Dose 2 | Doses 3-10 |
| 25 | 10 | mg/kg | 10 mg/kg (BID) | | |
| | | (BID) | | | |
| 26 | 25 | mg/kg | 25 mg/kg (BID) | | |
| | | (BID) | | | |
| 27 | 25/5 | mg/kg | 25 mg/kg | 5 mg/kg | 5 mg/kg (BID) |
| 28 | 10/2 | mg/kg | 10 mg/kg | 2 mg/kg | 2 mg/kg (BID) |
| 29 | 50/10 | mg/kg | 50 mg/kg | 10 mg/kg | 10 mg/kg (BID) |
| 30 | 30/6 | mg/kg | 30 mg/kg | 6 mg/kg | 6 mg/kg (BID) |
| 31 | 25/5 | mg/kg | 25 mg/kg | | 5 mg/kg (QD) |
| | | (QD) | | | |
| 32 | 50/10 | mg/kg | 50 mg/kg | | 10 mg/kg (QD) |
| | | (QD) | | | |
| 33 | 25 | mg/kg | 25 mg/kg | | 25 mg/kg (QD) |
| | | (QD) | | | |
| 34 | 10 | mg/kg | 10 mg/kg | | 10 mg/kg (QD) |
| | | (QD) | | | |
| 35 | 30/10 | mg/kg | 30 mg/kg | 10 mg/kg | 10 mg/kg (BID) |
| | | (BID) | | | |
| 36 | 40/20 | mg/kg | 40 mg/kg | 20 mg/kg | 20 mg/kg (BID) |
| | | (BID) | | | |
| 37 | 60/40 | mg/kg | 60 mg/kg | 40 mg/kg | 40 mg/kg (BID) |
| | | (BID) | | | |

| | | | |
|---|---|---|---|
| 38 | 40 | mg/kg | 40 mg/kg (QD) |
| | | (QD) | |
| 39 | 60 | mg/kg | 60 mg/kg (QD) |
| | | (QD) | |

Some embodiments described herein relate to a method for ameliorating or treating a pneumovirus infection that can include administering a first dosage of compound (A), or a pharmaceutically acceptable salt thereof, and administering multiple separate second dosages of compound (A), or a pharmaceutically acceptable salt thereof, to a patient suffering from the pneumovirus infection, wherein the first dosage and the multiple separate second dosages are provided according to a regimen selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 in Tables 1, 2, and/or 3. Other embodiments described herein relate to using a first dosage compound (A), or a pharmaceutically acceptable salt thereof, and multiple separate second dosages of compound (A), or a pharmaceutically acceptable salt thereof, in the manufacture of a medicament for ameliorating and/or treating a pneumovirus infection in a patient suffering from the pneumovirus infection, wherein the first dosage and the multiple separate second dosages are provided according to a regimen selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 in Tables 1, 2, and/or 3. Still other embodiments described herein relate to a first dosage of compound (A), or a pharmaceutically acceptable salt thereof, and multiple separate second dosages of compound (A), or a pharmaceutically acceptable salt thereof, that can be used for ameliorating and/or treating a pneumovirus infection in a patient suffering from the pneumovirus infection, wherein the first dosage and the multiple separate second dosages are provided according to a regimen selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 in Tables 1, 2, and/or 3.

Compound (A), or a pharmaceutically acceptable salt thereof, can be formulated into various pharmaceutical forms for administration purposes. Additionally, various routes are suitable for providing compound (A), or a pharmaceutically acceptable salt thereof. In some embodiments, compound (A), or a pharmaceutically acceptable salt thereof, can be provided in an oral dosage form. Any orally acceptable dosage form including, but not limited to, capsules, tablets, pills, powders, granules, emulsions, microemulsions, suspensions (e.g., aqueous suspensions), syrups, elixirs, or solutions can be used to provide compound (A), or a pharmaceutically acceptable salt thereof. Liquid dosage forms for oral administration include, but are not limited to, pharmaceutically acceptable emulsions, microemulsions, solutions, suspensions, syrups and elixirs. Solid dosage forms for oral administration include capsules (for example, soft and hard-filled gelatin capsules), tablets, pills, powders, and granules. The oral dosage forms can be prepared using methods known to those skilled in the art and may contain additional materials such as pharmaceutically acceptable excipient(s) or carrier(s). In some embodiments, compound (A), or a pharmaceutically acceptable salt thereof, can be provided in an oral suspension.

Compound (A), or a pharmaceutically acceptable salt thereof, can be used in combination with one or more anti-RSV agents. One suitable anti-RSV agent is GS-5806 (N-(2-((S)-2-(5-((S)-3-Aminopyrrolidin-1-yl)-6-methylpyrazolo[1,5-a]pyrimidin-2-yl)piperidine-1-carbonyl)-4-chlorophenyl)methanesulfonamide), or a pharmaceutically acceptable salt thereof, (Gilead Sciences). GS-5806 is a RSV fusion inhibitor that can be given orally. As with compound (A), or a pharmaceutically acceptable salt thereof, GS-5806, or a pharmaceutically acceptable salt thereof, can be given at various dosages, frequency and length of time.

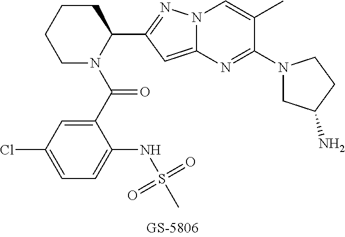 [SMILES: Cc1cn2nc([C@@H]3CCCCN3C(=O)c3cc(Cl)ccc3NS(C)(=O)=O)cc2nc1N1CC[C@H](N)C1]

Examples of suitable amounts of GS-5806, or a pharmaceutically acceptable salt thereof, include, but are not limited to, the following embodiments. In some embodiments, GS-5806, or a pharmaceutically acceptable salt thereof, can be administered to a patient suffering from RSV in an amount in the range of 75 mg to 100 mg, in combination with compound (A), or a pharmaceutically acceptable salt thereof. In other embodiments, GS-5806, or a pharmaceutically acceptable salt thereof, can be administered to a patient suffering from RSV in an amount in the range of 75 mg to 125 mg, in combination with compound (A), or a pharmaceutically acceptable salt thereof. In still other embodiments, GS-5806, or a pharmaceutically acceptable salt thereof, can be administered to a patient suffering from RSV in an amount in the range of 5 mg to 10 mg, in combination with compound (A), or a pharmaceutically acceptable salt thereof. In yet still other embodiments, GS-5806, or a pharmaceutically acceptable salt thereof, can be administered to a patient suffering from RSV in an amount in the range of 2.5 mg to 8 mg, in combination with compound (A), or a pharmaceutically acceptable salt thereof. In some embodiments, GS-5806, or a pharmaceutically acceptable salt thereof, can be administered to a patient suffering from RSV in an amount in the range of 10 mg to 75 mg, in combination with compound (A), or a pharmaceutically acceptable salt thereof. In other embodiments, GS-5806, or a pharmaceutically acceptable salt thereof, can be administered to a patient suffering from RSV in an amount in the range of 25 mg to 50 mg, in combination with compound (A), or a pharmaceutically acceptable salt thereof. In still other embodiments, GS-5806, or a pharmaceutically acceptable salt thereof, can be administered to a patient suffering from RSV in an amount in the range of 150 mg to 250 mg, in combination with compound (A), or a pharmaceutically acceptable salt thereof. In yet still other embodiments, GS-5806, or a pharmaceutically acceptable salt thereof, can be administered to a patient suffering from RSV in an amount in the range of 125 mg to 225 mg, in combination with compound (A), or a pharmaceutically acceptable salt thereof.

In some embodiments, GS-5806, or a pharmaceutically acceptable salt thereof, can be administered to a patient suffering from RSV in an amount in the range of 0.5 mg/kg to 10 mg/kg, in combination with compound (A), or a pharmaceutically acceptable salt thereof. In other embodiments, GS-5806, or a pharmaceutically acceptable salt thereof, can be administered to a patient suffering from RSV in an amount in the range of 1 mg/kg to 7 mg/kg, in combination with compound (A), or a pharmaceutically acceptable salt thereof. In still other embodiments, GS-5806, or a pharmaceutically acceptable salt thereof, can be administered to a patient suffering from RSV in an amount in the range of 1.5 mg/kg to 5 mg/kg, in combination with compound (A), or a pharmaceutically acceptable salt thereof.

As with compound (A), a first dosage of GS-5806, or a pharmaceutically acceptable salt thereof, can be administered, followed by several separate second dosages of GS-5806, or a pharmaceutically acceptable salt thereof. Suitable amounts of GS-5806, or a pharmaceutically acceptable salt thereof, for the first and second dosages are provided herein. In some embodiments, the first dosage of GS-5806, or a pharmaceutically acceptable salt thereof, can be provided in multiple dosages. The multiple dosages can be taken together at a first time period. Alternatively, at least one dosage form of the multiple dosages of the first dosage can be taken at a first time period and at least one dosage form of the multiple dosage forms of the first dosage can be taken at a second time period (for example, twice daily).

Examples of suitable regimens using GS-5806 that can be used in combination with any of the regimens described herewith with respect to compound (A), or a pharmaceutically acceptable salt thereof, include those provided in Table 4. The amounts in Table 4 are for GS-5806, or a pharmaceutically acceptable salt thereof.

TABLE 4

| | | | | Period of |
|---|---|---|---|---|
| | | | | Time for |
| Example | First Dosage | Period of Time | Each Second | Second |
| Regimen | (Day 1) | for First Dosage | Dosage | Dosages |
| 1 | 50 mg | 1 day | 25 mg (daily) | 4 days |
| 2 | 100 mg | 1 day | 5 mg (daily) | 4 days |
| 3 | 10 mg | 1 day | 5 mg (daily) | 4 days |
| 4 | 50 mg | 1 day | 25 mg (daily) | 2 days |
| 5 | 200 mg^{a} | 1 day | | |

^{a}can be given in a single dosage form or multiple dosage forms (e.g., 4 × 50 mg)

The order of administration of the compounds in a combination therapy (for example, a compound (A) and GS-5806, or a pharmaceutically acceptable salt of the foregoing) can vary. In some embodiments, compound (A), or a pharmaceutically acceptable salt thereof, can be administered prior to all compounds of the combination therapy. In other embodiments, compound (A), or a pharmaceutically acceptable salt thereof, can be administered prior to at least one compound of the combination therapy. In still other embodiments, compound (A), or a pharmaceutically acceptable salt thereof, can be administered concomitantly with one or more compound(s) of the combination therapy. In yet still other embodiments, compound (A), or a pharmaceutically acceptable salt thereof, can be administered subsequent to the administration of at least one compound of the combination therapy. In some embodiments, compound (A), or a pharmaceutically acceptable salt thereof, can be administered subsequent to the administration of all other compounds of the combination therapy.

In some embodiments, a combination of compound (A) and GS-5806, or a pharmaceutically acceptable salt of the foregoing, can result in an additive effect. In some embodiments, a combination of compound (A) and GS-5806, or a pharmaceutically acceptable salt of the foregoing, can result in a synergistic effect. In some embodiments, a combination of compound (A) and GS-5806, or a pharmaceutically acceptable salt of the foregoing, can result in a strongly synergistic effect. In some embodiments, a combination of compound (A) and GS-5806, or a pharmaceutically acceptable salt of the foregoing, is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of each compound is determined individually.

A potential advantage of utilizing a combination of compound (A) and GS-5806, or a pharmaceutically acceptable salt of the foregoing, may be a reduction in the required amount(s) of the compound(s) that is effective in treating RSV, as compared to the amount required to achieve same therapeutic result when the compound(s), is administered as monotherapy. For example, the amount of compound (A) and/or GS-5806, or a pharmaceutically acceptable salt of the foregoing, in a combination described herein can be less compared to the amount of compound (A) and/or GS-5806, or a pharmaceutically acceptable salt of the foregoing, needed to achieve the same viral load reduction when administered as a monotherapy. Another potential advantage of utilizing a combination of compound (A) and GS-5806, or a pharmaceutically acceptable salt of the foregoing, is that the use of two or more compounds having different mechanisms of action can create a higher barrier to the development of resistant viral strains compared to the barrier when a compound is administered as monotherapy. Additional advantages of utilizing a combination of compound (A) and GS-5806, or a pharmaceutically acceptable salt of the foregoing, may include little to no cross resistance between the compounds of the combination; different routes for elimination; little to no overlapping toxicities; little to no significant effects on cytochrome P450; and/or little to no pharmacokinetic interactions between the compounds of the combination.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

As used herein, the terms “prevent” and “preventing,” mean lowering the efficiency of viral replication and/or inhibiting viral replication to a greater degree in a patient who receives the compound compared to a patient who does not receive the compound. Examples of forms of prevention include prophylactic administration to a patient who has been or may be exposed to an infectious agent, such as a pneumovirus (e.g., RSV).

As used herein, a “patient” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the patient can be an adult human (18 years or older). In other embodiments, the patient can be child (>1-17 years). In still other embodiments, the patient can be an infant (1 year and younger). In yet still other embodiments, the patient can be a pediatric patient, wherein the term “pediatric” is used as understood by those skilled in the art. For example, pediatrics patients include infants, children and adolescents.

The terms “therapeutically effective amount” and “effective amount” are used to indicate an amount of an active compound, or pharmaceutical agent, that elicits the biological or medicinal response indicated. For example, an effective amount of compound can be the amount needed to prevent, alleviate or ameliorate symptoms of disease or prolong the survival of the patient being treated This response may occur in a tissue, system, animal or human and includes alleviation of the signs or symptoms of the disease being treated. Determination of an effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

Various indicators for determining the effectiveness of a method for treating a RSV viral infection are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, and/or other indicator of disease response.

In some embodiments, an effective amount of compound (A) and/or compound (B), or a pharmaceutically acceptable salt of the foregoing, is an amount that is effective to reduce viral titers to undetectable levels, for example, less than 1.7 log₁₀ plaque forming units equivalents (PFUe)/mL, or less than 0.3 log₁₀ plaque forming units equivalents (PFUe)/mL. In some embodiments, an effective amount of compound (A) and/or compound (B), or a pharmaceutically acceptable salt of the foregoing, is an amount that is effective to reduce viral load compared to the viral load before administration of compound (A), or a pharmaceutically acceptable salt thereof. For example, the viral load is measure before administration of compound (A), or a pharmaceutically acceptable salt thereof, and again several hours after receiving the initial dosage of compound (A), or a pharmaceutically acceptable salt thereof (for example, 60 hours after receiving the initial dosage of compound (A), or a pharmaceutically acceptable salt thereof). In some embodiments, compound (A) and/or compound (B), or a pharmaceutically acceptable salt of the foregoing, can be an amount that is effective to reduce viral load to lower than 1.7 log₁₀ (PFUe)/mL, or lower than 0.3 log₁₀ (PFUe)/mL. In some embodiments, an effective amount of compound (A) and/or compound (B), or a pharmaceutically acceptable salt of the foregoing, is an amount that is effective to achieve a reduction in viral titer in the serum of the patient in the range of about 1.5-log to about a 2.5-log reduction, about a 3-log to about a 4-log reduction, or a greater than about 5-log reduction compared to the viral load before administration of compound (A), or a pharmaceutically acceptable salt thereof. For example, the viral load is measure before administration of compound (A), or a pharmaceutically acceptable salt thereof, and several hours after receiving the initial dosage of compound (A), or a pharmaceutically acceptable salt thereof (for example, 60 hours after receiving the initial dosage of compound (A), or a pharmaceutically acceptable salt thereof).

In some embodiments, compound (A) and/or compound (B), or a pharmaceutically acceptable salt of the foregoing, can result in at least a 1, 2, 3, 4, 5, 10, 15, 20, 25, 50, 75, 100-fold or more reduction in the replication of RSV relative to pre-treatment levels in a patient, as determined several hours after receiving the initial dosage of compound (A), or a pharmaceutically acceptable salt thereof (for example, 60 hours after receiving the initial dosage of compound (A), or a pharmaceutically acceptable salt thereof). In some embodiments, compound (A) and/or compound (B), or a pharmaceutically acceptable salt of the foregoing, can result in a reduction of the replication of RSV relative to pre-treatment levels in the range of about 2 to about 5 fold, about 10 to about 20 fold, about 15 to about 40 fold, or about 50 to about 100 fold. In some embodiments, compound (A) and/or compound (B), or a pharmaceutically acceptable salt of the foregoing, can result in a reduction of RSV replication in the range of 1 to 1.5 log, 1.5 log to 2 log, 2 log to 2.5 log, 2.5 to 3 log, 3 log to 3.5 log or 3.5 to 4 log more reduction of RSV replication compared to the reduction of RSV reduction achieved by ribavirin (Virazole®), or may achieve the same reduction as that of ribavirin (Virazole®) therapy in a shorter period of time, for example, in one day, two days, three days, four days, or five days, as compared to the reduction achieved after 5 days of ribavirin (Virazole®) therapy.

In some embodiments, an effective amount of compound (A) and/or compound (B), or a pharmaceutically acceptable salt of the foregoing, is an amount that is effective to achieve an undetectable level of viral RNA in less than 5 days (120 hours) after the initial administration of the first dosage. In some embodiments, an effective amount of compound (A) and/or compound (B), or a pharmaceutically acceptable salt of the foregoing, is an amount that is effective to achieve an undetectable level of viral RNA in less than 3 days (72 hours) after the initial administration of the first dosage.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. Additionally, all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### EXAMPLES

### Example 1

### Treatment Regimens

Healthy adults received one of the following dosing regimens or placebo over 5 days using the human RSV challenge model.

| Regimen | First Dosage | Second Dosages | |
|---|---|---|---|
| 1 | 750 mg (single dosage) | 150 mg | N = 7 |
| 2 | 750 mg (single dosage) | 500 mg | N = 8 |

| | | |
|---|---|---|
| 3 | 375 mg | N = 8 |
| 4 | Placebo | N = 12 |

Patients were given an intranasal inoculation of RSV-A Memphis 37b challenge virus. Administration of compound (A), or a pharmaceutically acceptable salt thereof, began approximately 12 hours after confirmation of RSV infection as determined by the presence of RSV RNA in nasopharyngeal washes. The test compound was administered as an oral-liquid suspension, wherein the drug vehicle was methyl cellulose and sterile water. The placebo was the drug vehicle without the test compound. Second dosages were started 12 hours after administration of the first dosage, and the remaining second dosages were provided in approximate 12 hour intervals. Nasal washes were collected twice daily approximately 36 to 48 hours after RSV inoculation until Day 12. Viral load was detected and quantified from the aliquots of the nasal wash samples using tissue infectivity plaque assays and PCR. (See DeVincenzo, J. P., et al., Am. J. Respir. Crit. Care. Med. (2010) 182(10):1305-1314) Patients returned for two follow-up visits on Day 16 (±2 days) and Day 28 (±2 days) post-challenge inoculation.

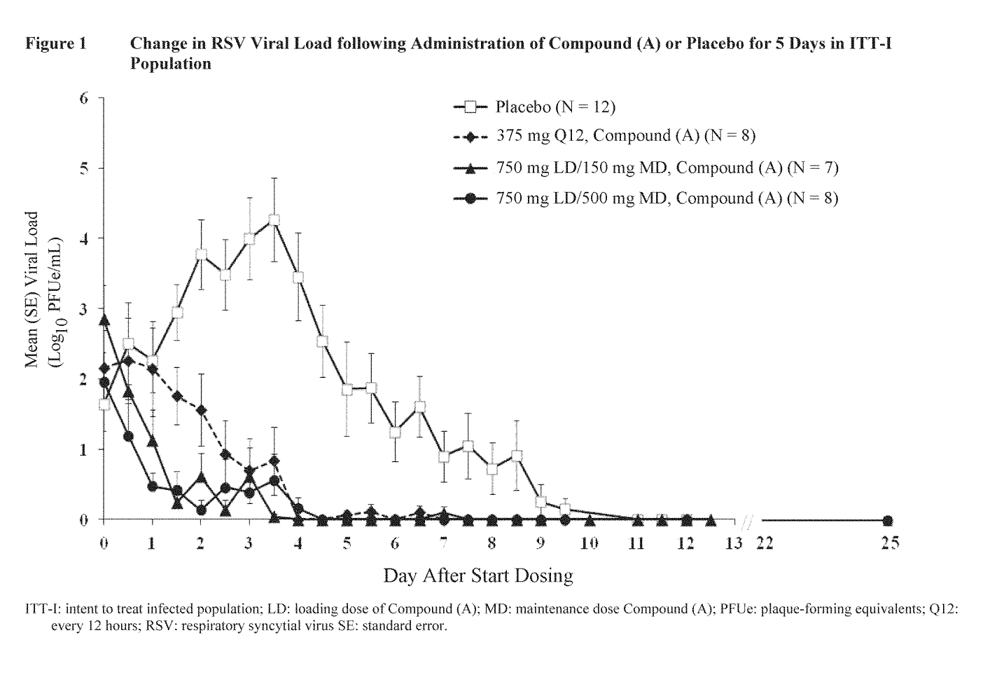

As shown in FIG. 1, all regimens with Compound (A), or a pharmaceutically acceptable salt thereof, resulted in the marked reduction in RSV viral load compared to the placebo. The placebo group exhibited a logarithmic increase in RSV RNA with a peak viral load at approximately Day 3.5 following the start of dosing. At Day 12, all patient treated with compound (A), or a pharmaceutically acceptable salt thereof, had undetectable RSV RNA. In contrast, the patients receiving the placebo had a mean RSV RNA of 0.52 log₁₀ plaque forming units equivalents (PFUe)/mL on Day 12. At both Day 16 and 28, RSV RNA remained undetectable in the patients who received compound (A), or a pharmaceutically acceptable salt thereof. Those patients who received 750 mg of compound (A), or a pharmaceutically salt thereof, on Day 1 had a multi-log reduction of plaque forming units equivalents (PFUe/mL) within the first 24 hours. Additionally, no patient discontinued treatment during the study and no clinically significant laboratory abnormalities were observed. Thus, compound (A), or a pharmaceutically acceptable salt thereof, provides a significant advancement for treating RSV.

### Example 2

### Treatment Regimens in Adult Patients

Within the clinical development program, a range of doses and durations are evaluated in adults with a RSV infection using a compound described herein (for example, compound (A), or a pharmaceutically acceptable salt thereof). For example, patients receive one of the following orally administered dosing regimens over 5-7 days in a randomized clinical trial.

| Dose Regimen | | | |
|---|---|---|---|
| (mg) | Dose 1 | Dose 2 | Doses 3-10 |
| 750/500 mg | 750 mg | 500 mg | 500 mg (BID) |
| 750/150 mg | 750 mg | 150 mg | 150 mg (BID) |
| 500/150 mg | 500 mg | 150 mg | 150 mg (BID) |

### Example 3

### Treatment Regimens in Pediatric Patients

Within the clinical development program, a range of doses and durations are evaluated in infants and children with a RSV infection using a compound described herein (for example, compound (A), or a pharmaceutically acceptable salt thereof). For example, patients receive one of the following orally administered dosing regimens over 5-7 days in a randomized clinical trial.

| Dose Regimen | | | |
|---|---|---|---|
| (mg/kg) | Dose 1 | Dose 2 | Doses 3-10 |
| 25/5 mg/kg | 25 mg/kg | 5 mg/kg | 5 mg/kg (BID) |
| 10/2 mg/kg | 10 mg/kg | 2 mg/kg | 2 mg/kg (BID) |
| 50/10 mg/kg | 50 mg/kg | 10 mg/kg | 10 mg/kg (BID) |
| 40/20 mg/kg | 40 mg/kg | 20 mg/kg | 20 mg/kg (BID) |
| 60/40 mg/kg | 60 mg/kg | 40 mg/kg | 40 mg/kg (BID) |

### Example 4

### Treatment Regimens in Pediatric Patients

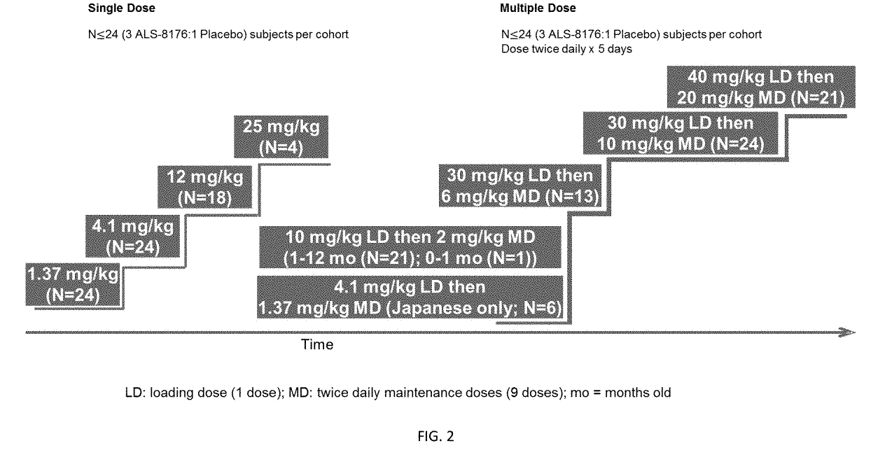

One hundred fifty eight otherwise healthy pediatric patients hospitalized with RSV infection were recruited in a randomized, double-blind, placebo-controlled, SAD (single ascending dose) and MAD (multiple ascending dose) study. The patients were randomized into nine SAD, MAD regimens. Subjects not given the placebo were administered lumicitabine or compound (A), a prodrug of the nucleoside analog AL-8112 (otherwise referenced herein as compound (C), the triphosphate of which is a potent inhibitor of the RSV polymerase. The cohort size and dose for the first SAD cohort was prespecified. The makeup of all other cohorts was determined by an Independent Data Monitoring Committee (IDMC) that regularly reviewed data. FIG. 2 provides the single and multiple doses used in the study as well as approximate numbers of subjects dosed. Safety and PK data were collected for 7 (SAD) and 28 (MAD) days post randomization. PK was analyzed using non-linear mixed effect modeling.

Part 1 (single ascending dose): Patients received a single dose of 1 of 4 ascending doses of compound (A), (1.37 mg/kg, 4.1 mg/kg, 12 mg/kg and 25 mg/kg) or placebo. Patients received compound (A) for a 7-day consecutive period.

Part 2 (multiple ascending dosage): Patients received a loading dosage followed by nine maintenance dosages, or placebo. Patients received compound (A) for a 5-day consecutive period. The maintenance dosages were administered twice daily. In the twice daily dosage regimen, the first maintenance dosage was given 8-18 hours after the loading dosage (dosage 1).

| Dose Regimen | Loading Dose | Maintenance Dose |
|---|---|---|
| (mg/kg) | (Dosage 1) | (Dosages 2-10) |
| 4.1/1.37 mg/kg | 4.1 mg/kg | 1.37 mg/kg (BID) |
| 10/2 mg/kg | 10 mg/kg | 2 mg/kg (BID) |
| 30/6 mg/kg | 30 mg/kg | 6 mg/kg (BID) |
| 30/10 mg/kg | 30 mg/kg | 10 mg/kg (BID) |
| 40/20 mg/kg | 40 mg/kg | 20 mg/kg (BID) |
| 60/40 mg/kg | 60 mg/kg | 40 mg/kg (BID) |

Seven serious adverse events (AEs) were reported in the SAD (phlebitis, tachycardia, respiratory failure) and MAD (lymphadenitis, bronchiolitis, neutropenia, pneumonia/respiratory failure), which were considered unlikely related to study drug (except neutropenia: possibly related). Two subjects discontinued study drug due to AEs (neutropenia, pneumonia/respiratory failure (both serious AEs)) and all treatment-emergent AEs (N=178) were mild (N=138) or moderate (N=32) except 7 events:

pneumonia/respiratory failure (N=3), anemia, and neutropenia (N=3), which were more severe. Blinded AEs reported in ≥5 pts (descending order of frequency) were: diarrhea, diaper rash, vomiting, thrombocytosis, aspartate transaminase increase, eczema, rash. No concerning trends (e.g., by dose, age) in AEs, laboratories, vital signs, physical examinations or electrocardiograms were identified in the blinded data or IDMC reports.

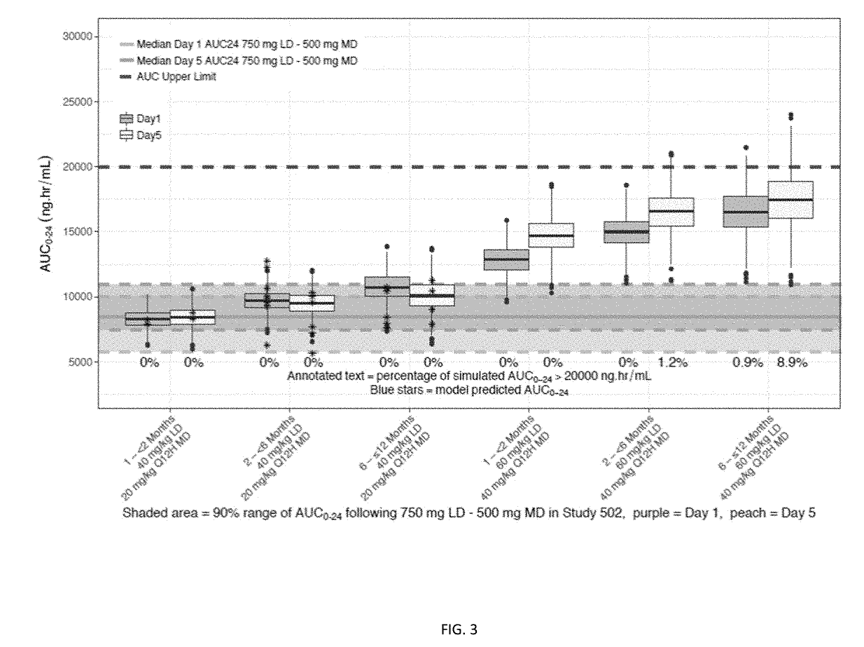

A PK model was developed integrating adult and pediatric data to aid calculation of PK parameters and project efficacious doses. For pediatric dose and exposure projections three age cohorts were used: 1-<2 months, 2-<6 month and 6-<12 months. Apparent clearance increased with dose, creatinine clearance and body weight (using allometric principles). Maturation functions were applied for age dependent changes in Carboxylesterase 1 abundance on bioavailability and kidney function on renal elimination. AL-8112 (also referenced herein as Compound (C)) plasma exposures increased in a less than dose proportional manner with increasing ALS-8176 doses; no accumulation was present. Actual AL-8112 plasma concentration data in conjunction with PK modeling, indicate that doses of 40/20 mg/kg and 60/40 mg/kg result targeted antiviral plasma AUC₀₋₂₄ of AL-8112 8,000 to 20,000 ng*h/mL (FIG. 3).

Interim results from the ongoing blinded study in otherwise healthy infants hospitalized with RSV infection demonstrate that single and multiple lumicitabine doses up to 25 mg/kg (single dose) and 60/40 mg/kg (multiple dose), respectively are well tolerated with low incidence of SAEs, treatment discontinuations, or severe TEAEs for an acutely ill, hospitalized patent population.

### Example 5

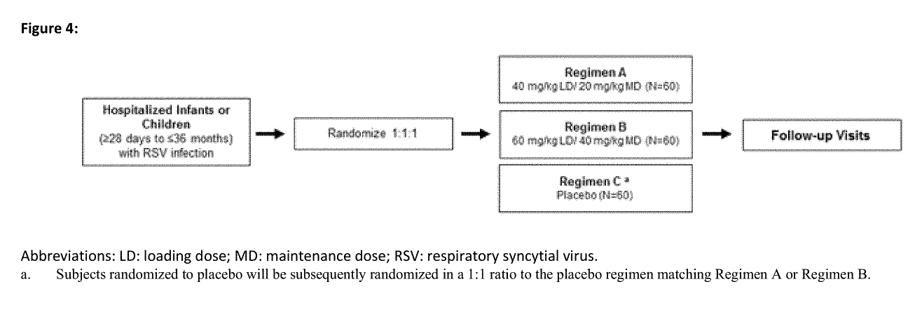

The antiviral activity, clinical outcomes, safety, tolerability, and pharmacokinetics of orally administered lumicitabine regimens are evaluated in a Phase 2, randomized, double-blind, placebo-controlled study of hospitalized infants and children aged 28 days to 36 months infected with RSV. The pediatric patients are randomized into one of several arms as shown in the schematic of FIG. 4.

### Example 6

### Combination Studies

RSV with Renilla Reporter

RSV expressing Renilla luciferase (A2-RL-line19F) are generated by Dr. Martin Moore of Emory University, Atlanta, Ga., USA. The in vitro viral kinetics of A2-RL-line19F is similar to that of wild type RSV (See Hotard, A. L., Virology (2012) 434(1):129-136).

Host cell HEp-2 is purchased from ATCC (Cat. #CCL-23) and cells are cultured in DMEM/Ham's F-12 50/50 1× containing L-glutamine and 15 mM HEPES (Mediatech, Cat. #10-092-CM). The medium is further supplemented with 5% (v/v) FBS (Mediatech, Cat. #35-010-CV) and 1% (v/v) penicillin/streptomycin (Mediatech, Cat. #30-002-CI). HEp-2 cells are maintained at 37° C. in a humidified 5% CO₂ atmosphere.

### Drug Treatment and Viral Dosing

To determine the effect of a combination of compounds, the following procedure is followed. On the first day, 20,000 HEp-2 cells are plated per well in a 96-well plate. On the following day, test articles are solubilized in 100% DMSO (for chemicals) or 1×PBS (for biologics) to 200× the desired final testing concentration. Subsequently, Compound (A), or a pharmaceutically acceptable salt thereof, is serially diluted (1:3) to 9 distinct concentrations “horizontally” in a 96-well plate, and the second test compound is serially diluted (1:3) to 7 distinct concentrations “vertically” in 96-well plate. The serially diluted 200× test articles are then diluted 1:10 into cell culture media to generate 20× test articles. A 5 μL aliquot of the 20× test articles is added in a checkerboard fashion to the cells with 90 μL existing media. Space is also allotted for titrations of each of the compounds alone to be used as reference controls. After 12 hour pre-incubation of test articles, A2-RL-line19F at an MOI of 0.5 is added to the plate and further incubated for 2 days at 37° C. in a 5% CO₂.

### Determination of Anti-RSV Activity

The Renilla Luciferase Assay System (Promega, Cat. # E2820) is used to measure anti-RSV replicon activity. Assay plates were set up as stated above. Luminescence is recorded using a Perkin Elmer multilabel counter Victor3V.

### Cell Viability Assay

Promega CellTiter-Glo Luminescent Cell Viability Assay, Cat. #G7572) is used to measure cell viability. The CellTiter-Glo® Luminescent Cell Viability Assay is a homogeneous method to determine the number of viable cells in culture based on quantitation of the adenosine triphosphate (ATP) present, which signals the presence of metabolically active cells. Assay plates are set up in the same format the anti-RSV assay, except that no virus is added to the cell viability assay. A 100-μL aliquot of CellTiter-Glo reagent is added to each well and incubated at room temperature for 8 minutes. Luminescence is recorded using a Perkin Elmer multilabel counter Victor3V.

### Data Analysis

Each experiment is performed at N=5 for both anti-RSV activity and cell viability. Mean percent inhibition of the replicon values from the 5 experiments is generated and for anti-RSV activity, it is analyzed using two drug interaction analysis models, Isobologram Analysis and/or Prichard's Model.

### Isobologram Analysis

The effects of drug-drug combinations are evaluated by the Loewe additivity model in which the experimental data are analyzed using CalcuSyn (Biosoft, Ferguson, Mo.), a computer program based on the method of Chou and Talalay. The combination index (CI) value and the isobologram for each experimental combination are calculated. CI values of <1, 1, and >1 indicate synergy, additive effect, and antagonism, respectively. Under the synergy category, CI<0.1 is considered very strong synergism; CI 0.1-0.3 strong synergism; CI 0.3-0.7 synergism and CI 0.7-0.85 moderate synergism. The isobologram analysis, which graphically represents additive, synergistic, and antagonistic drug effects, is also used to model the interaction of antiviral activities. In this representation, an effective concentration (EC) value of one drug is plotted on one axis and corresponding EC value of a second drug is plotted on the second axis; the line connecting these two points represents the amount of each drug in a combination that would be required to reach the equivalent EC value, given that their effects are additive.

### Prichard's Model (MacSynergy II)

MacSynergy II software is kindly provided by Dr. M. Prichard (University of Michigan). This program allows the three-dimensional examination of drug interactions of all data points generated from the checkerboard combination of two inhibitors with Bliss-Independence model. Confidence bounds are determined from replicate data. If the 95% confidence limits (CL) do not overlap the theoretic additive surface, then the interaction between the two drugs differs significantly from additive. The volumes of synergy or antagonism can be determined and graphically depicted in three dimensions and represent the relative quantity of synergism or antagonism per change in the two drug concentrations. Synergy and antagonism volumes are based on the Bliss independence model, which assumes that both compounds act independently on different targets. A set of predicted fractional responses faAB under the Bliss independence model is calculated as faAB=faA+faB−faA·faB with faA and faB representing the fraction of possible responses, e.g. % inhibition, of compounds A and B at amounts dA and dB, respectively, and describes the % inhibition of a combination of compounds A and B at amount (dA+dB). If faAB>faA+faB−faA·faB then we have Bliss synergy; if faAB<faA+faB−faA·faB then we have Bliss antagonism. The 95% synergy/antagonism volumes are the summation of the differences between the observed inhibition and the 95% confidence limit on the prediction of faAB under the Bliss independence model. Table 5 shows the volumes and corresponding volume descriptions for the results of the Bliss Independence Analysis. MacSynergy II is used for data analysis.

TABLE 5
MacSynergy II Volume Descriptions

| Volume (μM² %) | Volume Description |
|---|---|
| <25 | Additive |
| 25-50 | Minor synergism |
| 50-100 | Significant synergism |
| >100 | Strong synergism |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A method for treating respiratory syncytial virus (RSV) in a human pediatric patient, said method comprising administering to a human pediatric patient in need of treatment of RSV, compound (A) 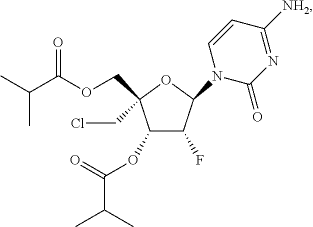 [SMILES: CC(C)C(=O)OC[C@@]1(CCl)O[C@@H](n2ccc(N)nc2=O)[C@H](F)[C@@H]1OC(=O)C(C)C]
or a pharmaceutically acceptable salt thereof, according to a dosing regimen comprising a first dose of compound (A), or a pharmaceutically acceptable salt thereof, in the range of 30 mg/kg-70 mg/kg; and one or more second doses of compound (A), or a pharmaceutically acceptable salt thereof, in the range of 10 mg/kg-50 mg/kg.

2. The method of claim 1, wherein the dosing regimen comprises a first dose in the range of 40 mg/kg-60 mg/kg; and one or more second doses in the range of 20 mg/kg-40 mg/kg.

3. The method of claim 1, wherein the first dose is 60 mg/kg and the second dose is 40 mg/kg.

4. The method of claim 1, wherein the first dose is 40 mg/kg and the second dose is 20 mg/kg.

5. The method of claim 1, wherein the first dose and the one or more second doses are administered BID.

6. The method of claim 1, wherein the dosing regimen comprises nine second doses of compound (A) or a pharmaceutically acceptable salt thereof.

7. The method of claim 1, wherein the dosing regimen spans a period from three to seven days.

8. The method of claim 7, wherein the dosing regimen spans a period from five to six days.

9. The method of claim 1, wherein the first dose is a loading dose (LD); and the one or more second doses are maintenance doses (MD).

10. The method of claim 1, wherein the human pediatric patient is a child.

11. The method of claim 1, wherein the human pediatric patient is an infant.

12. The method of claim 1, wherein compound (A), or the pharmaceutically acceptable salt thereof, is administered orally to the human pediatric patient.

13. The method of claim 12, wherein compound (A), or the pharmaceutically acceptable salt thereof, is administered in a liquid formulation.

14. The method of claim 13, wherein the liquid formulation is a suspension.

15. The method of claim 1, wherein the pediatric patient is infected with RSV type A.

16. The method of claim 1, wherein the pediatric patient is infected with RSV type B.

17. A method for treating respiratory syncytial virus (RSV), said method comprising administering to a human pediatric patient infected with RSV, compound (A) 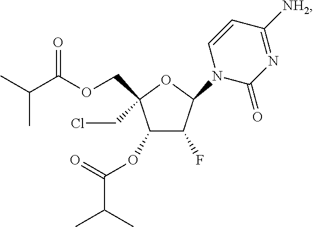 [SMILES: CC(C)C(=O)OC[C@@]1(CCl)O[C@@H](n2ccc(N)nc2=O)[C@H](F)[C@@H]1OC(=O)C(C)C]
or a pharmaceutically acceptable salt thereof, according to a dosing regimen comprising a first dose at 60 mg/kg followed by nine second doses at 40 mg/kg BID.

18. The method of claim 17, wherein compound (A) or the pharmaceutically acceptable salt thereof is administered orally to the human pediatric patient.

19. The method of claim 17, wherein the human pediatric patient is a child.

20. The method of claim 17, wherein the human pediatric patient is an infant.

21. The method of claim 17, wherein compound (A), or the pharmaceutically acceptable salt thereof, is administered in a liquid formulation.

22. The method of claim 21, wherein the liquid formulation is a suspension.

23. The method of claim 17, wherein the patient is infected with RSV type A.

24. The method of claim 17, wherein the patient is infected with RSV type B.

25. A method for treating respiratory syncytial virus (RSV), said method comprising administering to a human pediatric patient infected with RSV, compound (A) 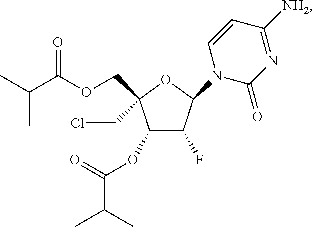 [SMILES: CC(C)C(=O)OC[C@@]1(CCl)O[C@@H](n2ccc(N)nc2=O)[C@H](F)[C@@H]1OC(=O)C(C)C]
or a pharmaceutically acceptable salt thereof, according to a dosing regimen comprising a first dose at 40 mg/kg followed by nine second doses at 20 mg/kg BID.

26. The method of claim 25, wherein compound (A) or the pharmaceutically acceptable salt thereof is administered orally to the human pediatric patient.

27. The method of claim 25, wherein the human pediatric patient is a child.

28. The method of claim 25, wherein the human pediatric patient is an infant.

29. The method of claim 25, wherein compound (A), or the pharmaceutically acceptable salt thereof, is administered in a liquid formulation.

30. The method of claim 29, wherein the liquid formulation is a suspension.

31. The method of claim 25, wherein the patient is infected with RSV type A.

32. The method of claim 25, wherein the patient is infected with RSV type B.

33. A method for treating respiratory syncytial virus (RSV) in a human pediatric patient, said method comprising orally administering to the human pediatric patient in need of treatment for RSV, compound (A) 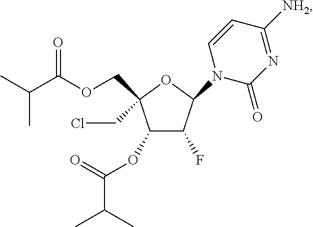 [SMILES: CC(C)C(=O)OC[C@@]1(CCl)O[C@@H](n2ccc(N)nc2=O)[C@H](F)[C@@H]1OC(=O)C(C)C]
or a pharmaceutically acceptable salt thereof, according to a dosing regimen of a dose from about 30 mg/kg-about 70 mg/kg of compound (A), or a pharmaceutically acceptable salt thereof, BID, wherein the dosing regimen spans a period of three to seven days.

34. The method of claim 33, wherein the dose is from about 40 mg/kg-about 60 mg/kg of compound (A), or a pharmaceutically acceptable salt thereof, BID.

35. The method of claim 33, wherein the dosing regimen has ten doses of compound (A) or a pharmaceutically acceptable salt thereof.

36. The method of claim 33, wherein the dosing regimen is 60 mg/kg of compound (A) or a pharmaceutically acceptable salt thereof, BID.

37. The method of claim 33, wherein the dosing regimen is 40 mg/kg of compound (A) or a pharmaceutically acceptable salt thereof, BID.

38. The method of claim 33, wherein the human pediatric patient is a child.

39. The method of claim 33, wherein the human pediatric patient is an infant.

40. The method of claim 33, wherein compound (A), or the pharmaceutically acceptable salt thereof, is administered in a suspension.

41. A method of treating RSV in a human pediatric patient, said method comprising orally administering to the human pediatric patient infected with RSV, compound (A), or a pharmaceutically acceptable salt thereof, according to a dosing regimen comprising a loading dose followed by nine maintenance doses BID, wherein said loading dose and maintenance doses are administered at levels sufficient to effect a mean AUC₀₋₂₄ of compound (C): 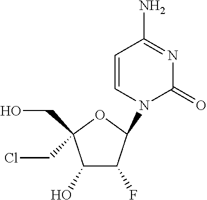 [SMILES: Nc1ccn([C@@H]2O[C@@](CO)(CCl)[C@@H](O)[C@H]2F)c(=O)n1]
in said human pediatric patient in the range of about 8,000 ng*hr/mL to about 20,000 ng*hr/mL following administration of compound (A) or the pharmaceutically acceptable salt thereof.

42. The method of claim 41, wherein said loading dose and maintenance doses are administered at levels sufficient to effect a mean AUC₀₋₂₄ of compound (C) in said human pediatric patient in the range of about 12,000 ng*hr/mL to about 20,000 ng*hr/mL following administration of compound (A) or the pharmaceutically acceptable salt thereof.

43. The method of claim 41, wherein said loading dose and maintenance doses are administered at levels sufficient to effect a mean AUC₀₋₂₄ of compound (C) in said human pediatric patient in the range of about 12,000 ng*hr/mL to about 19,000 ng*hr/mL following administration of compound (A) or the pharmaceutically acceptable salt thereof.

44. The method of claim 41, wherein said loading dose and maintenance doses are administered at levels sufficient to effect a mean AUC₀₋₂₄ of compound (C) in said human pediatric patient in the range of about 8,000 ng*hr/mL to about 12,000 ng*hr/mL following administration of compound (A) or the pharmaceutically acceptable salt thereof.

45. The method of claim 41, wherein the loading dose is selected from the range of 30 mg/kg to 70 mg/kg and the maintenance dose is selected from the range of 10 mg/kg to 50 mg/kg.

46. The method of claim 45, wherein the loading dose is selected from the range of 40 mg/kg to 60 mg/kg and the maintenance dose is selected from the range of 20 mg/kg to 40 mg/kg.

47. The method of claim 46, wherein the loading dose is 40 mg/kg and the maintenance dose is 20 mg/kg.

48. The method of claim 46, wherein the loading dose is 60 mg/kg and the maintenance dose is 40 mg/kg.

49. The method of claim 41, wherein the human pediatric patient is a child.

50. The method of claim 41, wherein the human pediatric patient is an infant.

51. The method of claim 41, wherein compound (A), or the pharmaceutically acceptable salt thereof, is administered in a liquid formulation.

52. The method of claim 51, wherein the liquid formulation is a suspension.

53. The method of claim 41, wherein the human pediatric patient is infected with RSV type A.

54. The method of claim 41, wherein the human pediatric patient is infected with RSV type B.
